(19) United States
(12) Patent Application Publication
(10) Pub. No.: US 2012/0201885 A1
(43) Pub. Date: Aug. 9, 2012

(54) PHARMACEUTICAL COMPOSITIONS OF COMBINATIONS OF DIPEPTIDYL PEPTIDASE-4 INHIBITORS WITH PIOGLITAZONE

(75) Inventors: Nicholas Birringer, Philadelphia, PA (US); Christopher T. John, Perkiomenville, PA (US); Zhen Liu, Chalfont, PA (US); Adam Procopio, Cinnaminson, NJ (US); Bhagwant Rege, Rockville, MD (US)

(73) Assignee: MERCK SHARP & DOHME CORP

(21) Appl. No.: 13/501,252

(22) PCT Filed: Oct. 12, 2010
(86) PCT No.: PCT/US10/52225

§ 371 Date: Apr. 11, 2012

(30) Foreign Application Priority Data
Oct. 23, 2009 (US) 61254299

(51) Int. Cl. A61K 9/20; A61K 31/675; A61P 3/10; A61K 31/513; A61K 31/4439; A61K 9/28; A61K 31/4985

(52) U.S. Cl. 424/465; 424/464; 424/474; 514/81; 514/249; 514/274; 514/342

(57) ABSTRACT

This invention relates to a bilayer pharmaceutical compositions comprising fixed-dose combinations of a dipeptidyl peptidase-4 inhibitor and pioglitazone, methods of preparing such pharmaceutical compositions, and methods of treating Type 2 diabetes with such pharmaceutical compositions.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral antidiabetic monotherapy. For many patients, these regimens do not sufficiently control glycaemia during long-term treatment, leading to a requirement for combination therapy within several years following diagnosis. However, co-prescription of two or more oral antidiabetic drugs may result in treatment regimens that are complex and difficult for many patients to follow. Combining two or more oral antidiabetic agents into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral antidiabetic agents include Glucovance™ (metformin and glyburide), Avandamet™ (metformin and rosiglitazone), and Metaglip™ (metformin and glipizide).

Currently sitagliptin phosphate monohydrate and pioglitazone HCl are each available as separate tablets for the treatment of type 2 diabetes. Treatment of type 2 diabetes with the combination of sitagliptin phosphate monohydrate and pioglitazone HCl, acting on different targets, has superior efficacy relative to treatment with either sitagliptin phosphate monohydrate or pioglitazone HCl alone. This invention provides a pharmaceutical composition comprising sitagliptin, or a pharmaceutically acceptable salt thereof, and pioglitazone HCl in a single bilayer tablet for superior efficacy and stability in the treatment of type 2 diabetes. The separate pioglitazone HCl layer in the bilayer tablet of the present invention provides the benefit of increased stability of the pioglitazone layer and a reduction in the disproportionation of pioglitazone HCl in the pioglitazone layer of the bilayer tablet.

Pioglitazone hydrochloride (ACTOS®) is a thiazolidinedione PPAR-γ agonist used in the management of type 2 diabetes mellitus (also known as non-insulin dependent diabetes mellitus or adult onset diabetes) primarily by decreasing insulin resistance. Pharmacological studies indicate that pioglitazone hydrochloride improves sensitivity to insulin in muscle and adipose tissue, inhibits hepatic gluconeogenesis, and improves glycemic control while reducing circulating insulin levels.

Dipeptidyl peptidase-4 (DPP-4) inhibitors represent a novel class of agents that are being developed for the treatment or improvement in glycemic control in patients with Type 2 diabetes. Specific DPP-4 inhibitors currently in clinical trials for the treatment of Type 2 diabetes include sitagliptin phosphate (MK-0431), vildagliptin (LAF-237), saxagliptin (BMS-47718), alogliptin (X), carmegliptin (X), melogliptin (X), dutogliptin (X), denagliptin (X), linagliptin (X), P93/01 (Prosidion), SYR322 (Takeda), GSK 823093, Roche 0730699, TS021 (Taisho), E3024 (Eisai), and PHX-1149 (Phenomix). For example, oral administration of vildagliptin or sitagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) H.-U. Demuth, et al., “Type 2 diabetes—Therapy with dipeptidyl peptidase IV inhibitors, Biochim. Biophys. Acta, 1751: 33-44 (2005) and (2) K. Augustyns, et al., “Inhibitors of praline-specific dipeptidyl peptidases: DPP IV inhibitors as a novel approach for the treatment of Type 2 diabetes,” Expert Opin. Ther. Patents, 15: 1387-1407 (2005).

Sitagliptin phosphate having structural formula I below is the dihydrogen phosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

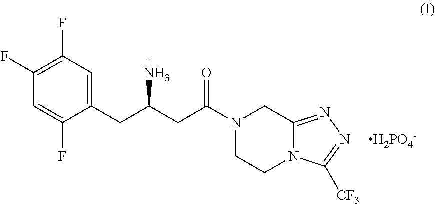

In one embodiment, sitagliptin phosphate is in the form of a crystalline anhydrate or monohydrate. In a class of this embodiment, sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in international patent publication WO 2005/0031335 published on Jan. 13, 2005. For a review on sitagliptin phosphate (MK-0431) including its synthesis and pharmacological properties, reference is made to the following publications: (1) C. F. Deacon, “MK-431,” Curr. Opin. Invest. Drugs, 6: 419-426 (2005) and (2) “MK-0431”, Drugs of the Future,” 30: 337-343 (2005).

Vildagliptin (LAF-237) is the generic name for (S)-1-[(3-hydroxy-1-adamantyl)amino]acetyl-2-cyano-pyrrolidine having structural formula II. Vildagliptin is specifically disclosed in U.S. Pat. No. 6,166,063, the contents of which are hereby incorporated by reference in their entirety.

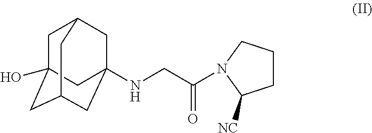

Saxagliptin (BMS-47718) is a methanoprolinenitrile of structural formula III below. Saxagliptin is specifically disclosed in U.S. Pat. No. 6,395,767, the contents of which are hereby incorporated by reference in their entirety.

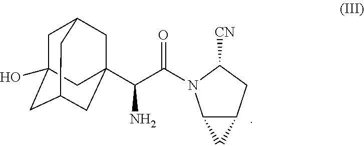

Alogliptin (SYR-322) is a DP-IV inhibitor under investigation for the treatment of type 2 diabetes of structural formula IV below:

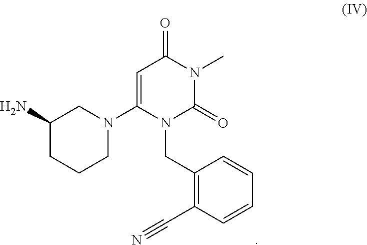

Other DP-IV inhibitors useful in the formulation of the present invention include, but are not limited to: alogliptin, carmegliptin, melogliptin, dutogliptin, denagliptin, linagliptin, saxagliptin and vildagliptin.

The present invention provides for pharmaceutical compositions of a fixed-dose combination of a dipeptidyl peptidase-4 inhibitor (DPP-4 inhibitor) and pioglitazone which are prepared by dry or wet processing methods. The pharmaceutical compositions of the present invention provide for immediate release of the two active pharmaceutical ingredients. In one embodiment the pharmaceutical compositions of the present invention are in the dosage form of a tablet, and, in particular, a film-coated tablet.

The present invention also provides a process to prepare pharmaceutical compositions of a fixed-dose combination of a DPP-4 inhibitor and pioglitazone by dry and wet processing methods. The dry processing methods include dry compression and dry granulation, and the wet processing methods include wet granulation, such as fluid bed granulation and high-shear granulation. In one embodiment, the DPP-4 inhibitor layer is prepared by direct compression, and the pioglitazone layer is prepared by fluid bed granulation.

Another aspect of the present invention provides methods for the treatment of Type 2 diabetes by administering to a host in need of such treatment a therapeutically effective amount of a pharmaceutical composition of the present invention.

These and other aspects will become readily apparent from the detailed description which follows.

### SUMMARY OF THE INVENTION

The present invention is directed to novel pharmaceutical compositions comprising fixed dose combinations of a dipeptidyl peptidase-4 inhibitor (DPP-4 inhibitor) and pioglitazone, or pharmaceutically acceptable salts of each thereof, methods of preparing such pharmaceutical compositions, and methods of treating Type 2 diabetes with such pharmaceutical compositions. In particular, the invention is directed to pharmaceutical compositions comprising fixed-dose combinations of sitagliptin phosphate and pioglitazone hydrochloride.

### DETAILED DESCRIPTION OF THE INVENTION

One aspect of the present invention is directed to dosage forms for the medicinal administration of a fixed-dose combination of a dipeptidyl peptidase-4 inhibitor (DPP-4 inhibitor) and pioglitazone. Such dosage forms may be in the powder or solid format including, but not limited to, tablets, capsules, and sachets. A particular solid dosage form relates to tablets comprising a fixed-dose combination of a DPP-4 inhibitor and pioglitazone hydrochloride (also known as [(±)-5-[[4-[2-(5-ethyl-2-pyridinyl)ethoxy]phenyl]-methyl]-2,4-]thiazolidinedione monohydrochloride).

In a particular aspect of the present invention, the pharmaceutical compositions in the form of a bilayer tablet comprise: (a) a first layer comprising a dipeptidyl peptidase-4 inhibitor, or a pharmaceutically acceptable salt thereof; and (b) a second layer comprising pioglitazone hydrochloride. In one embodiment of the present invention, the first bilayer additionally comprises one or more excipients selected from the group consisting of: (i) a diluent; (ii) a disintegrant; and (iii) a lubricant. In another embodiment of the present invention, the second bilayer additionally comprises one or more excipients selected from the group consisting of (i) a diluent, (ii) a disintegrant; (iii) a binding agent; and (iv) a lubricant. In another embodiment of the present invention, the pharmaceutical compositions may also contain one or more surfactants or wetting agents; and one or more antioxidants.

In another embodiment of this aspect of the invention, the DPP-4 inhibitor is selected from the group consisting of sitagliptin, vildagliptin, saxagliptin, P93/01, SYR322, GSK 823093, Roche 0730699, TS021, E3024, and PHX-1149. In a class of this embodiment the DPP-4 inhibitor is alogliptin, carmegliptin, melogliptin, dutogliptin, denagliptin, linagliptin, sitagliptin, vildagliptin, or saxagliptin. In a subclass of this class, the DPP-4 inhibitor is sitagliptin.

A preferred pharmaceutically acceptable salt of sitagliptin is the dihydrogen phosphate salt of structural formula I above (sitagliptin phosphate). A preferred form of the sitagliptin dihydrogen phosphate salt is the crystalline monohydrate (sitagliptin phosphate monohydrate) disclosed in WO 2005/0031335.

The preparation of sitagliptin and pharmaceutically acceptable salts thereof is disclosed in U.S. Pat. No. 6,699,871, the contents of which are herein incorporated by reference in their entirety. The preparation of sitagliptin phosphate monohydrate is disclosed in international patent publication WO 2005/0031335 published on Jan. 13, 2005, the contents of which are herein incorporated by reference in their entirety.

The dosage strength of the DPP-4 inhibitor for incorporation into the pharmaceutical compositions of the present invention is an amount from about 1 milligram to about 250 milligrams of the active moiety. A preferred dosage strength of the DPP-4 inhibitor is an amount from about 25 milligrams to about 200 milligrams of the active moiety. Discrete dosage strengths are the equivalent of 25, 50, 75, 100, 150, and 200 milligrams of the DPP-4 inhibitor active moiety. By “active moiety” is meant the free base form of the DPP-4 inhibitor as an anhydrate.

The unit dosage strength of sitagliptin free base anhydrate (active moiety) for inclusion into the fixed-dose combination pharmaceutical compositions of the present invention is 25, 50, 75, 100, 150, or 200 milligrams. A preferred dosage strength of sitagliptin is 50 or 100 milligrams. An equivalent amount of sitagliptin phosphate monohydrate to the sitagliptin free base anhydrate is used in the pharmaceutical compositions, namely, 32.13, 64.25, 96.38, 128.5, 192.75, and 257 milligrams, respectively.

The dosage strength of pioglitazone for incorporation into the pharmaceutical compositions of the present invention is an amount from about 1 milligram to about 100 milligrams of the active moiety. A preferred dosage strength of pioglitazone is an amount from about 15 milligrams to about 45 milligrams of the active moiety. Discrete dosage strengths are the equivalent of 15, 30, and 45 milligrams of the pioglitazone active moiety. By “active moiety” is meant the free base form of pioglitazone.

The unit dosage strength of the pioglitazone (active moiety) for inclusion into the fixed-dose combination pharmaceutical compositions of the present invention is 15 milligrams, 30 milligrams, and 45 milligrams. An equivalent amount of pioglitazone hydrochloride to the pioglitazone free base (or active moiety) is used in the pharmaceutical compositions, namely, 16.53 milligrams, 33.06 milligrams and 49.59 milligrams, respectively. These unit dosage strengths of pioglitazone represent the dosage strengths approved in the U.S. for marketing to treat Type 2 diabetes.

Specific embodiments of dosage strengths for sitagliptin and pioglitazone in the fixed-dose combinations of the present invention are the following:
(1) 50 milligrams of sitagliptin (equivalent to 64.25 milligrams of sitagliptin phosphate monohydrate) and 15 milligrams pioglitazone (equivalent to 16.53 milligrams of pioglitazone hydrochloride);
(2) 50 milligrams of sitagliptin (equivalent to 64.25 milligrams of sitagliptin phosphate monohydrate) and 30 milligrams pioglitazone (equivalent to 33.06 milligrams of pioglitazone hydrochloride);
(3) 50 milligrams of sitagliptin (equivalent to 64.25 milligrams of sitagliptin phosphate monohydrate) and 45 milligrams pioglitazone (equivalent to 49.59 milligrams of pioglitazone hydrochloride);
(4) 100 milligrams of sitagliptin (equivalent to 128.5 milligrams of sitagliptin phosphate monohydrate) and 15 milligrams pioglitazone (equivalent to 16.53 milligrams of pioglitazone hydrochloride);
(5) 100 milligrams of sitagliptin (equivalent to 128.5 milligrams of sitagliptin phosphate monohydrate) and 30 milligrams pioglitazone (equivalent to 33.06 milligrams of pioglitazone hydrochloride); and
(6) 100 milligrams of sitagliptin (equivalent to 128.5 milligrams of sitagliptin phosphate monohydrate) and 45 milligrams pioglitazone (equivalent to 49.59 milligrams of pioglitazone hydrochloride).

The pharmaceutical compositions of the present invention are prepared by dry and wet processing methods. In one embodiment the pioglitazone HCl layer is prepared by wet processing methods. In a class of this embodiment, the pioglitazone HCl layer is prepared by wet granulation methods. With wet granulation either high-shear granulation or fluid-bed granulation may be used. In another class of this embodiment, the pioglitazone HCl layer is prepared by fluid-bed granulation. Fluid bed granulation processing has the advantage of affording tablets with higher diametric strength. The wet processing methods enhance the chemical stability of pioglitazone HCl. In particular the wet processing methods minimize the disproportionation pioglitazone HCl to the pioglitazone free base, and result in consistent dissolution of pioglitazone HCl over the shelf life of the combination when stored in appropriate packaging material. In another embodiment the DPP-4 layer is prepared by dry processing methods. In a class of this embodiment, the DPP-4 layer is prepared by direct compression. Additionally, using a bilayer tablet with a separate pioglitazone HCl layer containing a disintegrant, such as crospovidone, further reduces the disproportionation of pioglitazone HCl and further increases stability of the tablet.

The pharmaceutical compositions obtained by dry and wet processing methods may be compressed into tablets, encapsulated, or metered into sachets.

The pharmaceutical compositions contain one or more lubricants or glidants. Examples of lubricants include magnesium stearate, calcium stearate, stearic acid, sodium stearyl fumarate, hydrogenated castor oil, and mixtures thereof. In one embodiment, the lubricant is magnesium stearate or sodium stearyl fumarate, or a mixture thereof. In another embodiment, the lubricant is magnesium stearate. In another embodiment, the lubricant is sodium stearyl fumarate. Examples of glidants include colloidal silicon dioxide, calcium phosphate tribasic, magnesium silicate, and talc.

The pharmaceutical compositions of the present invention optionally contain one or more binding agents. Embodiments of binding agents include hydroxypropylcellulose (HPC), hydroxypropylmethyl cellulose (HPMC), hydroxyethyl cellulose, starch 1500, polyvinylpyrrolidone (povidone), and co-povidone. In one embodiment, the binding agent is polyvinylpyrrolidone. In another embodiment, the binding agent is hydroxypropylcellulose (HPC). In another embodiment, the binding agent is hydroxypropylcellulose (HPC) in solution. In another embodiment, the binding agent is hydroxypropylcellulose (HPC) in an aqueous solution. In another embodiment, the binding agent is hydroxypropylcellulose (HPC).

The pharmaceutical compositions of the present invention may also optionally contain one or more diluents. Examples of diluents include mannitol, sorbitol, anhydrous dibasic calcium phosphate, lactose monohydrate, dibasic calcium phosphate dihydrate, microcrystalline cellulose, and powdered cellulose. In one embodiment the diluent is selected from: mannitol, anhydrous dibasic calcium phosphate, lactose monohydrate and microcrystalline cellulose, or a mixture of any two, three or four thereof. In one embodiment the diluent is selected from: anhydrous dibasic calcium phosphate, lactose monohydrate and microcrystalline cellulose, or a mixture of any two or three thereof. In another embodiment, the diluent is microcrystalline cellulose. Microcrystalline cellulose is available from several suppliers and includes Avicel, Avicel PH 101, Avicel PH 102, Avicel, PH 103, Avicel PH 105, and Avicel PH 200, manufactured by the FMC Corporation. In another embodiment, the diluent is mannitol. In another embodiment, the diluent is a mixture of microcrystalline cellulose and mannitol. In another embodiment, the diluent is a 2:1 to 1:2 mixture of microcrystalline cellulose to mannitol. In another embodiment, the diluent is microcrystalline cellulose, mannitol and anhydrous dibasic calcium phosphate. In another embodiment, the diluent is microcrystalline cellulose or mannitol or anhydrous dibasic calcium phosphate. In another embodiment, the diluent is microcrystalline cellulose and anhydrous dibasic calcium phosphate. In another embodiment, the diluent is mannitol and anhydrous dibasic calcium phosphate. In another embodiment, the diluent is anhydrous dibasic calcium phosphate. In another embodiment of the present invention, the diluent is lactose monohydrate.

The pharmaceutical compositions of the present invention may also optionally contain a disintegrant. The disintegrant may be one of several modified starches, modified cellulose polymers, or polycarboxylic acids, such as croscarmellose sodium, sodium starch glycolate, polacrillin potassium, carboxymethylcellulose calcium (CMC Calcium), and crospovidone. In one embodiment, the disintegrant is selected from: polacrillin potassium, carboxymethylcellulose calcium (CMC Calcium), and crospovidone. In another embodiment, the disintegrant is crospovidone and croscarmellose sodium. In another embodiment, the disintegrant is crospovidone. In another embodiment, the disintegrant is croscarmellose sodium.

The pharmaceutical compositions of the present invention may also optionally contain one or more surfactants or wetting agents. The surfactant may be anionic, cationic, or neutral. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral surfactants include glyceryl monooleate, polyoxyethylene sorbitan fatty acid esters, polyvinyl alcohol, and sorbitan esters. Embodiments of wetting agents include poloxamer, polyoxyethylene alkyl ethers, polyoxyethylene castor oil derivatives, and polyoxyethylene stearates.

The pharmaceutical compositions of the present invention may also optionally contain an anti-oxidant which may be added to the formulation to impart chemical stability. The anti-oxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is BHT or BHA.

Preferred dosage forms for the pharmaceutical compositions of the present invention are tablets which are prepared by compression methods. Such tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coating agent is Opadry® which is a formulated powder blend provided by Colorcon. Embodiments of Opadry® useful in the present invention include, but are not limited to, Opadry® I (HPC/HPMC), Opadry® 20A18334, Opadry® II, Opadry® II HP (PVA-PEG), or another suitable Opacity® suspension (such as polyvinyl alcohol, polyethylene glycol, titanium dioxide, and talc, with or without colorants).

Finally, a sweetening agent and/or flavoring agent may be added if desired.

In one embodiment of the present invention, the pharmaceutical composition comprises:

(a) a first layer comprising about 20 to 45% by weight of a dipeptidyl peptidase-4 inhibitor, or a pharmaceutically acceptable salt thereof; and
(b) a second layer comprising about 7 to 24% by weight of pioglitazone hydrochloride.

In a class of this embodiment, the first layer additionally comprises one or more excipients selected from the group consisting of: (i) a diluent; (ii) a disintegrant; and (iii) a lubricant. In a subclass of this class, the first layer additionally comprises one or more excipients selected from the group consisting of (i) two diluents; (ii) a disintegrant; and (iii) two lubricants.

In another class of this embodiment, the first layer additionally comprises one or more excipients selected from the group consisting of: (i) about 40-80% by weight of a diluent; (ii) about 0.5-6% by weight of a disintegrant; and (iii) about 0.75-10% by weight of a lubricant. In a subclass of this class, the first layer additionally comprises one or more excipients selected from the group consisting of: (i) about 40-80% by weight of two diluents; (ii) about 0.5-6% by weight of a disintegrant; and (iii) about 0.75-10% by weight of two lubricants.

In another class of this embodiment, the first layer additionally comprises one or more excipients selected from the group consisting of: (i) about 20-40% by weight of a first diluent; (ii) about 20-40% of a second diluent; (iii) about 0.5-6% by weight of a disintegrant; (iv) about 0.25-4% by weight of a first lubricant and (v) about 0.5-6% by weight of a second lubricant. In a subclass of this class, the first diluent is microcrystalline cellulose; the second diluent is anhydrous dibasic calcium phosphate; the disintegrant is croscarmellose sodium; the first lubricant is magnesium stearate; and the second lubricant is sodium stearyl fumarate.

In another class of this embodiment, the second layer additionally comprises one or more excipients selected from the group consisting of: (i) a diluent, (ii) a disintegrant; (iii) a binding agent; and (iv) a lubricant.

In another class of this embodiment, the second layer additionally comprises one or more excipients selected from the group consisting of: (i) about 60-80% by weight of a diluent; (ii) about 2-12% by weight of a disintegrant; and (iii) about 1-7% by weight of a binding agent; and (iv) about 0.25-4% by weight of a lubricant.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In a second embodiment of the present invention, the pharmaceutical composition comprises:

(a) a first layer comprising:

(i) about 20 to 45% by weight of a dipeptidyl peptidase-4 inhibitor, or a pharmaceutically acceptable salt thereof;

(ii) about 40-80% by weight of a diluent;

(iii) about 0.5-6% by weight of a disintegrant; and

(iv) about 0.75-10% by weight of a lubricant; and

(b) a second layer comprising:

(i) about 7 to 24% by weight of pioglitazone hydrochloride;

(ii) about 60-80% by weight of a diluent;

(iii) about 2-12% by weight of a disintegrant;

(iv) about 1-7% by weight of a binding agent, and

(v) about 0.25-4% by weight of a lubricant.

In another class of this embodiment, the first layer additionally comprises one or more excipients selected from the group consisting of: (i) about 40-80% by weight of two diluents; (ii) about 0.5-6% by weight of a disintegrant; and (iii) about 0.75-10% by weight of two lubricants.

In another class of this embodiment, the first layer additionally comprises one or more excipients selected from the group consisting of: (i) about 20-40% by weight of a first diluent; (ii) about 20-40% of a second diluent; (iii) about 0.5-6% by weight of a disintegrant; (iv) about 0.25-4% by weight of a first lubricant and (v) about 0.5-6% by weight of a second lubricant. In a subclass of this class, the first diluent is microcrystalline cellulose; the second diluent is anhydrous dibasic calcium phosphate; the disintegrant is croscarmellose sodium; the first lubricant is magnesium stearate; and the second lubricant is sodium stearyl fumarate.

In another class of this embodiment, the pharmaceutical composition comprises a first layer wherein the diluent is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof. In another class of this embodiment, the pharmaceutical composition comprises a first layer wherein the diluent is a mixture of microcrystalline cellulose and mannitol, or a mixture of microcrystalline cellulose and anhydrous dibasic calcium phosphate; the disintegrant is crospovidone or croscarmellose sodium; and the lubricant is a mixture of magnesium stearate and sodium stearyl fumarate.

In another class of this embodiment, the pharmaceutical composition comprises a first layer wherein the diluent is a mixture of microcrystalline cellulose and mannitol; the disintegrant is crospovidone; and the lubricant is a mixture of magnesium stearate and sodium stearyl fumarate.

In another class of this embodiment, the pharmaceutical composition comprises a first layer wherein the diluent is a mixture of microcrystalline cellulose and anhydrous dibasic calcium phosphate; the disintegrant is croscarmellose sodium; and the lubricant is a mixture of magnesium stearate and sodium stearyl fumarate.

In another class of this embodiment, the pharmaceutical composition comprises a second layer wherein the diluent is selected from the group consisting of: anhydrous dibasic calcium phosphate, lactose monohydrate, microcrystalline cellulose and mannitol, or a mixture thereof; the disintegrant is selected from the group consisting of: crospovidone and croscarmellose sodium, or a mixture thereof; the binding agent is hydroxypropyl cellulose; and the lubricant is selected from the group consisting of: magnesium stearate and sodium stearyl fumarate, or a mixture thereof. In a subclass of this class, the diluent selected from the group consisting of lactose monohydrate, microcrystalline cellulose and mannitol, or a mixture thereof; the disintegrant is selected from the group consisting of: crospovidone and croscarmellose sodium; the binding agent is hydroxypropyl cellulose; and the lubricant is selected from the group consisting of: magnesium stearate and sodium stearyl fumarate, or a mixture thereof. In another subclass of this class, the pharmaceutical composition comprises a second layer wherein the diluent is lactose monohydrate; the disintegrant is crospovidone; the binding agent is hydroxypropyl cellulose; and the lubricant is magnesium stearate. In another subclass of this class, the pharmaceutical composition comprises a second layer wherein the diluent is lactose monohydrate; the disintegrant is crospovidone; the binding agent is hydroxypropyl cellulose; and the lubricant is sodium stearyl fumarate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In a third embodiment of the present invention, the pharmaceutical composition comprises:

(a) a first layer comprising:

(i) about 25 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 1.5-7% by weight of a lubricant; and

(b) a second layer comprising:

(i) about 12 to 20% by weight of pioglitazone hydrochloride;

(ii) about 65-75% by weight of a diluent;

(iii) about 3-11% by weight of a disintegrant;

(iv) about 2-5% by weight of a binding agent; and

(v) about 0.5-2.5% by weight of a lubricant.

In a class of this embodiment, the pharmaceutical composition comprises: (a) a first layer comprising: (i) about 25 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 1.5-7% by weight of two lubricants.

In another class of this embodiment, the first layer additionally comprises one or more excipients selected from the group consisting of: (i) about 25-35% by weight of a first diluent; (ii) about 25-35% of a second diluent; (iii) about 1-4% by weight of a disintegrant; (iv) about 0.5-2% by weight of a first lubricant and (v) about 1-5% by weight of a second lubricant. In a subclass of this class, the first diluent is microcrystalline cellulose; the second diluent is anhydrous dibasic calcium phosphate; the disintegrant is croscarmellose sodium; the first lubricant is magnesium stearate; and the second lubricant is sodium stearyl fumarate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and mannitol, or a mixture of microcrystalline cellulose and anhydrous dibasic calcium phosphate; the disintegrant is croscarmellose sodium or crospovidone; and the lubricant is a mixture of magnesium stearate and sodium stearyl fumarate. In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In another class of this embodiment, the pharmaceutical composition comprises a second layer wherein the diluent is lactose monohydrate; the disintegrant is crospovidone; the binding agent is hydroxypropylcellulose; and the lubricant is magnesium stearate. In another class of this embodiment, the pharmaceutical composition comprises a second layer wherein the diluent is lactose monohydrate; the disintegrant is crospovidone; the binding agent is hydroxypropyl cellulose; and the lubricant is sodium stearyl fumarate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another class of the embodiments of the present invention, the pharmaceutical composition contains about 20 to 45% by weight of sitagliptin dihydrogen phosphate. In a subclass of this class, the composition contains about 25 to 35% of sitagliptin dihydrogen phosphate. In another subclass of this class, the composition contains about 32.12% of sitagliptin dihydrogen phosphate.

In another class of the embodiments of the present invention, the pharmaceutical composition contains about 25 to 45% by weight of sitagliptin, or a pharmaceutically acceptable salt thereof. In a subclass of this class, the composition contains about 25 to 35% of sitagliptin, or a pharmaceutically acceptable salt thereof. In another subclass of this class, the composition contains about 32.12% of sitagliptin, or a pharmaceutically acceptable salt thereof.

In another class of the embodiments of the present invention, the pharmaceutical composition contains about 7 to 24% by weight of pioglitazone HCl. In a subclass of this class, the composition contains about 12 to 20% of pioglitazone HCl. In another subclass of this class, the composition contains about 16.53% of pioglitazone HCl.

In another class of the embodiments of the present invention, the pharmaceutical composition contains about 7 to 24% by weight of pioglitazone, or a pharmaceutically acceptable salt thereof. In a subclass of this class, the composition contains about 12 to 20% of pioglitazone, or a pharmaceutically acceptable salt thereof. In another subclass of this class, the composition contains about 16.53% of pioglitazone, or a pharmaceutically acceptable salt thereof.

In another class of the embodiments of the present invention, the first layer of the pharmaceutical composition (the Sitagliptin layer) contains about 40 to 80% by weight of a diluent. In a subclass of this class, the composition contains about 50 to 70% of a diluent. In another subclass of this class, the composition contains about 61.88% of a diluent. In another subclass of this class, the composition contains about 60.88% of a diluent. In another subclass of this class, the composition contains about 20 to 40% of a first diluent; and contains about 20 to 40% of a second diluent. In another subclass of this class, the composition contains about 25 to 35% of a first diluent; and contains about 25 to 35% of a second diluent. In another subclass of this class, the composition contains about 30 to 31% of a first diluent; and contains about 30 to 31% of a second diluent. In another subclass of this class, the composition contains about 30.94% of a first diluent. In another subclass of this class, the composition contains about 30.94% of a second diluent. In another subclass of this class, the composition contains about 30.44% of a first diluent. In another subclass of this class, the composition contains about 30.44% of a second diluent. In another subclass of this class, the diluent is microcrystalline cellulose or mannitol. In another subclass of this class, the diluent is microcrystalline cellulose and mannitol. In another subclass of this class, the first diluent is microcrystalline cellulose and second diluent is mannitol. In another subclass of this class, the diluent is microcrystalline cellulose. In another subclass of this class, the diluent is mannitol. In another subclass of this class, the diluent is microcrystalline cellulose or anhydrous dibasic calcium phosphate. In another subclass of this class, the diluent is microcrystalline cellulose and anhydrous dibasic calcium phosphate. In another subclass of this class, the first diluent is microcrystalline cellulose and second diluent is anhydrous dibasic calcium phosphate.

In another class of the embodiments of the present invention, the second layer of the pharmaceutical composition (the Pioglitazone layer) contains about 60 to 80% by weight of a diluent. In a subclass of this class, the composition contains about 65 to 75% of a diluent. In another subclass of this class, the composition contains about 71-75% of a diluent. In another subclass of this class, the composition contains about 71.47% of a diluent. In another subclass of this class, the composition contains about 72.47% of a diluent. In another subclass of this class, the composition contains about 73.47% of a diluent. In another subclass of this class, the composition contains about 74.47% of a diluent. In another subclass of this class, the diluent is microcrystalline cellulose or mannitol. In another subclass of this class, the diluent is microcrystalline cellulose and mannitol. In another subclass of this class, the diluent is microcrystalline cellulose. In another subclass of this class, the diluent is mannitol. In another subclass of this class, the diluent is lactose monohydrate.

In another class of the embodiments of the present invention, the first layer of the pharmaceutical composition (the Sitagliptin layer) contains about 0.5-6% by weight of a disintegrant. In a subclass of this class, the composition contains about 1 to 4% of a disintegrant. In another subclass of this class, the composition contains about 2 to 3% of a disintegrant. In another subclass of this class, the composition contains about 2% of a disintegrant. In another subclass of this class, the composition contains about 3% of a disintegrant. In another subclass of this class, the disintegrant is croscarmellose sodium. In another subclass of this class, the disintegrant is crospovidone.

In another class of the embodiments of the present invention, the second layer of the pharmaceutical composition (the Pioglitazone layer) contains about 2 to 12% by weight of a disintegrant. In a subclass of this class, the composition contains about 3 to 11% of a disintegrant. In another subclass of this class, the composition contains about 3% of a disintegrant. In another subclass of this class, the composition contains about 5% of a disintegrant. In another subclass of this class, the composition contains about 6% of a disintegrant. In another subclass of this class, the composition contains about 8% of a disintegrant. In another subclass of this class, the composition contains about 10-11% of a disintegrant. In another subclass of this class, the disintegrant is crospovidone.

In another class of the embodiments of the present invention, the first layer of the pharmaceutical composition (the Sitagliptin layer) contains about 0.75 to 10% by weight of a lubricant. In a subclass of this class, the composition contains about 1.5 to 7% of a lubricant. In another subclass of this class, the composition contains about 4% of a lubricant. In another subclass of this class, the composition contains about 0.25 to 4% of a first lubricant; and contains about 0.5 to 6% of a second lubricant. In another subclass of this class, the composition contains about 0.5 to 2% of a first lubricant; and contains about 1 to 5% of a second lubricant. In another subclass of this class, the composition contains about 1% of a first lubricant; and contains about 3% of a second lubricant. In another subclass of this class, the lubricant is sodium stearyl fumarate or magnesium stearate. In another subclass of this class, the lubricant is sodium stearyl fumarate and magnesium stearate. In another subclass of this class, the lubricant is sodium stearyl fumarate. In another subclass of this class, the lubricant is magnesium stearate. In another class of this embodiment, the binding agent is hydroxypropylcellulose or polyvinylpyrrolidone, and the lubricant is sodium stearyl fumarate or magnesium stearate. In another class of this embodiment, the binding agent is hydroxypropylcellulose, and the lubricant is sodium stearyl fumarate. In another class of this embodiment, the binding agent is hydroxypropylcellulose, and the lubricant is magnesium stearate. In another class of this embodiment, the binding agent is hydroxypropylcellulose, and the lubricant is sodium stearyl fumarate and magnesium stearate. In another class of this embodiment, the binding agent is hydroxypropylcellulose, and the second lubricant is sodium stearyl fumarate and first lubricant is magnesium stearate.

In another class of the embodiments of the present invention, the second layer of the pharmaceutical composition (the Pioglitazone layer) contains about 0.25 to 4% by weight of a lubricant. In a subclass of this class, the composition contains about 0.5 to 2.5% of a lubricant. In a subclass of this class, the composition contains about 0.5 to 1.1% of a lubricant. In another subclass of this class, the composition contains about 1.05% of a lubricant. In another subclass of this class, the composition contains about 1% of a lubricant. In another subclass of this class, the composition contains about 0.5% of a lubricant. In another subclass of this class, the lubricant is sodium stearyl fumarate or magnesium stearate. In another subclass of this class, the lubricant is sodium stearyl fumarate and magnesium stearate. In another subclass of this class, the lubricant is sodium stearyl fumarate. In another subclass of this class, the lubricant is magnesium stearate. In another class of this embodiment, the binding agent is hydroxypropylcellulose or polyvinylpyrrolidone, and the lubricant is sodium stearyl fumarate or magnesium stearate. In another class of this embodiment, the binding agent is hydroxypropylcellulose, and the lubricant is sodium stearyl fumarate. In another class of this embodiment, the binding agent is hydroxypropylcellulose, and the lubricant is magnesium stearate.

In another class of the embodiments of the present invention, the second layer of the pharmaceutical composition (the Pioglitazone layer) contains about 1 to 7% by weight of a binding agent. In a subclass of this class, the composition contains about 2 to 5% of a binding agent. In another subclass of this class, the composition contains about 3 to 5% of a binding agent. In another subclass of this class, the composition contains about 3% of a binding agent. In another subclass of this class, the composition contains about 5% of a binding agent. In another subclass of this class, the binding agent is hydroxypropylcellulose or polyvinylpyrrolidone. In another subclass of this class, the binding agent is hydroxypropylcellulose. In another subclass of this class, the binding agent is hydroxypropylcellulose or polyvinylpyrrolidone, and the lubricant is sodium stearyl fumarate or magnesium stearate. In another subclass of this class, the binding agent is hydroxypropylcellulose, and the lubricant is sodium stearyl fumarate. In another subclass of this class, the binding agent is hydroxypropylcellulose, and the lubricant is magnesium stearate.

In further embodiments of the present invention, the pharmaceutical compositions are envisioned for commercial development:

### Tablets of 50 mg Dipeptidyl Peptidase-4 Inhibitor/15 mg Pioglitazone Potency

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of Pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of: croscarmellose sodium and crospovidone, or a mixture thereof; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

### Tablets of 50 mg Dipeptidyl Peptidase-4 Inhibitor/30 mg Pioglitazone Potency

For the first layer: about 32.12% by weight of the first layer of the dipeptidyl peptidase-4 inhibitor or a pharmaceutically acceptable salt thereof, about 60-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of Pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of: croscarmellose sodium and crospovidone, or a mixture thereof, the lubricant is selected from the group consisting of magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

### Tablets of 50 mg Dipeptidyl Peptidase-4 Inhibitor/45 mg Pioglitazone Potency

For the first layer: about 32.12% by weight of the first layer of the dipeptidyl peptidase-4 inhibitor or a pharmaceutically acceptable salt thereof; about 60-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of Pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of croscarmellose sodium and crospovidone, or a mixture thereof; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

### Tablets of 100 mg Dipeptidyl Peptidase-4 Inhibitor/15 mg Pioglitazone Potency:

For the first layer: about 32.12% by weight of the first layer of the dipeptidyl peptidase-4 inhibitor or a pharmaceutically acceptable salt thereof; about 60-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of Pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of: croscarmellose sodium and crospovidone, or a mixture thereof; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In another subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

### Tablets of 100 mg DIPEPTIDYL Peptidase-4 Inhibitor/30 mg Pioglitazone Potency:

For the first layer: about 32.12% by weight of the first layer of the dipeptidyl peptidase-4 inhibitor or a pharmaceutically acceptable salt thereof; about 60-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of Pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of: croscarmellose sodium and crospovidone, or a mixture thereof; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In another subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

### Tablets of 100 mg Dipeptidyl Peptidase-4 Inhibitor/45 mg Pioglitazone Potency:

For the first layer: about 32.12% by weight of the first layer of the dipeptidyl peptidase-4 inhibitor or a pharmaceutically acceptable salt thereof; about 60-62% by weight of the first layer of a diluent; about 2-3% by weight of the first layer of a disintegrant; and about 4% by weight of the first layer of a lubricant. For the second layer: about 16.53% by weight of the second layer of Pioglitazone HCl, about 71-75% by weight of the second layer of a diluent, about 3-11% by weight of the second layer of a disintegrant; about 0.5-1.5% by weight of the second layer of a lubricant; and about 3-5% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of microcrystalline cellulose, mannitol, lactose monohydrate, or a mixture thereof; the disintegrant is crospovidone; the lubricant is selected from the group consisting of: magnesium stearate, sodium stearyl fumarate, or a mixture thereof; and the binding agent is hydroxypropyl cellulose. In a subclass of this class, the diluent in the first layer is a mixture of microcrystalline cellulose and mannitol; the diluent in the second layer is lactose monohydrate; the lubricant in the first layer is a mixture of magnesium stearate and sodium stearyl fumarate; and the lubricant in the second layer is magnesium stearate. In another subclass the dipeptidyl peptidase-4 inhibitor is sitagliptin, or a pharmaceutically acceptable salt thereof.

Alternatively, for the first layer about 32.12% by weight of the first layer of the dipeptidyl peptidase-4 inhibitor or a pharmaceutically acceptable salt thereof; about 62% by weight of the first layer of a diluent; about 2% by weight of the first layer of a disintegrant; and about 4% by weight of the first layer of a lubricant. In the second layer: about 16.53% by weight of the second layer of pioglitazone HCl; about 74% by weight of the second layer of a diluent, about 6% by weight of the second layer of a disintegrant; about 3% by weight of the second layer of a binding agent; and about 0.5% by weight of the second layer of a lubricant. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof; the diluent is selected from the group consisting of: microcrystalline cellulose, anhydrous dibasic calcium phosphate, and lactose monohydrate, or a mixture thereof, the disintegrant is selected from the group consisting of: croscarmellose sodium and crospovidone, or a mixture thereof; the lubricant is selected from the group consisting of magnesium stearate, sodium stearyl fumarate, or a mixture thereof; and the binding agent is hydroxypropyl cellulose. In a subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is magnesium stearate; the disintegrant in the first layer is croscarmellose sodium; and the disintegrant in the second layer is crospovidone. In another subclass of this class, the diluent in the first layer is a mixture of microcrystalline cellulose and anhydrous dibasic calcium phosphate; the diluent in the second layer is lactose monohydrate; the lubricant in the first layer is a mixture of magnesium stearate and sodium stearyl fumarate; and the lubricant in the second layer is sodium stearyl fumarate; the disintegrant in the first layer is croscarmellose sodium; and the disintegrant in the second layer is crospovidone. In another subclass the dipeptidyl peptidase-4 inhibitor is sitagliptin, or a pharmaceutically acceptable salt thereof.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated. Substances which may be used for coating include hydroxypropylcellulose, hydroxypropylmethylcellulose, titanium dioxide, talc, sweeteners, colorants, and flavoring agents.

The term and symbol “% by weight” and “%” as used herein refers to the percentage by weight of the excipient and active ingredient (DPP-4 inhibitor or pioglitazone HCl) in each individual layer in the bilayer tablet, wherein the “individual layer” means the first layer or the second layer of the bilayer tablet.

In one embodiment the pharmaceutical compositions of the present invention are prepared by wet granulation (pioglitazone HCl layer) and dry processing (DPP-4 inhibitor layer). In one class of this embodiment, the pioglitazone HCl layer was prepared by fluid bed wet granulation. In another class of this embodiment, the DPP-4 layer was prepared by direct compression. Granulation is a process in which binding agent is added either through the granulating solution or through addition to the granulating bowl to form granules. The steps involved in the wet granulation and dry processing method comprise the following:

(1) Preparation of the first layer containing Sitagliptin phosphate:
(i) Sitagliptin phosphate, at least one diluent (such as microcrystalline cellulose and/or mannitol and/or anhydrous dibasic calcium phosphate), and at least one disintegrant (such as crospovidone and/or croscarmellose sodium) are blended in a suitable blender to form a blend;
(ii) the above blend is lubricated with at least one lubricant (such as magnesium stearate and/or sodium stearyl fumarate) in a suitable blender to form a lubricated powder blend;
(iii) the lubricated powder blend is roller compacted in a suitable roller compactor and the resulting ribbons are milled with in line rotary mill to give roller compacted granules; and
(iv) the roller compacted powder blend granules are lubricated with at least one lubricant (such as sodium stearyl fumarate and/or magnesium stearate) in a suitable blender.
   (2) Preparation of the second layer containing Pioglitazone HCl:
(i) Pioglitazone HCl, a diluent (such as lactose monohydrate), and half of at least one disintegrant (such as crospovidone) are mixed in fluid bed granulator to give a powder mixture;
(ii) a binding agent (such as hydroxypropyl cellulose (HPC)) is dissolved in water to give the binder solution for the binder solution spray;
(iii) the powder mixture in fluid bed granulator is granulated with the binder solution spray to give a granulation;
(iv) the granulation is dried in the fluid bed granulator;
(v) the dried granulation is de-lumped using a suitable mill to give de-lumped granules;
(vi) the de-lumped granules are mixed with the remaining half of the disintegrant (such as crospovidone and/or croscarmellose sodium) in a suitable blender to give a granulation lend; and
(vii) the above granulation lend is lubricated with at least one lubricant (such as magnesium stearate) in a suitable blender.
   (3) Preparation of the Bilayer Tablet via bilayer compression and coating:
(i) the Sitagliptin layer is used as the first layer in the bilayer press and tamped lightly;
(ii) the Pioglitazone layer is added and main compression force is applied to give bilayer tablets; and
(iii) the bilayer tablets are coated with Opadry I (HPC/HPMC).

A suitable processing method comprises the following steps:

(1) Preparation of the first layer containing Sitagliptin phosphate:
(i) sitagliptin phosphate, microcrystalline cellulose, mannitol and crospovidone are blended in a suitable blender to form a blend;
(ii) the above blend is lubricated with magnesium stearate in a suitable blender;
(iii) the lubricated powder blend is roller compacted in a suitable roller compactor and the resulting ribbons are milled with in line rotary mill to give roller compacted granules; and
(iv) the roller compacted granules are lubricated with sodium stearyl fumarate in a suitable blender.
   (2) Preparation of the second layer containing Pioglitazone HCl:
(i) Pioglitazone HCl, lactose monohydrate, and half of the crospovidone are mixed in fluid bed granulator to give a powder mixture;
(ii) hydroxypropyl cellulose (HPC) is dissolved in water to give the binder solution for the binder solution spray;
(iii) the powder mixture in fluid bed granulator is granulated with the binder solution spray to give a granulation;
(iv) the granulation is dried in the fluid bed granulator;
(v) the dried granulation is de-lumped using a suitable mill to give de-lumped granules;
(vi) the de-lumped granules are mixed with the remaining crospovidone in a suitable blender to give a granulation lend; and
(vii) the above granulation lend is lubricated with magnesium stearate in a suitable blender.
   (3) Preparation of the Bilayer Tablet via bilayer compression and coating:
(i) the Sitagliptin layer is used as the first layer in the bilayer press and tamped lightly;
(ii) the Pioglitazone layer is added and main compression force is applied to give bilayer tablets; and
(iii) the bilayer tablets are coated with Opadry I (HPC/HPMC).

Another suitable processing method comprises the following steps:

(1) Preparation of the first layer containing Sitagliptin:
(i) Sitagliptin phosphate, microcrystalline cellulose, anhydrous dibasic calcium phosphate and croscarmellose sodium are blended in a suitable blender; and
(ii) the above blend is lubricated with magnesium stearate and sodium stearyl fumarate in a suitable blender.
   (2) Preparation of the second layer containing Pioglitazone:
(i) Pioglitazone HCl, lactose monohydrate, and half of the crospovidone are mixed in fluid bed granulator to give a powder mixture;
(ii) hydroxypropyl cellulose (HPC) is dissolved in water to give the binder solution;
(iii) the powder mixture in fluid bed granulator is granulated with the binder solution spray to give a granulation;
(iv) the granulation is dried in the fluid bed granulator;
(v) the dried granulation is de-lumped using a suitable mill;
(vi) the de-lumped granules are mixed with the remaining crospovidone in a suitable blender to give a granulation lend; and
(vii) the above granulation lend is lubricated with magnesium stearate in a suitable blender.
   (3) Preparation of the Bilayer Tablet via bilayer compression and coating:
(i) the Sitagliptin layer is used as the first layer in the bilayer press and tamped lightly;
(ii) the Pioglitazone layer is added and main compression force is applied to give bilayer tablets;
(iii) the bilayer tablets are coated with Opadry I (HPC/HPMC).

Another suitable processing method comprises the following steps:

(1) Preparation of the first layer containing Sitagliptin:
(i) Sitagliptin phosphate, microcrystalline cellulose, anhydrous dibasic calcium phosphate and croscarmellose sodium are blended in a suitable blender; and
(ii) the above blend is lubricated with magnesium stearate and sodium stearyl fumarate in a suitable blender.
   (2) Preparation of the second layer containing Pioglitazone:
(i) Pioglitazone HCl, lactose monohydrate, and an intragranular portion of crospovidone (equal to 5% of the weight of the pioglitazone layer) are blended in a suitable blender and delumped using a suitable mill to give a blend;
(ii) hydroxypropyl cellulose (HPC) is dissolved in water 6% w/w to give a binder solution;
(iii) the blend is charged into a fluid bed granulator and granulated with the binder solution to give a granulation;
(iv) the granulation is dried;
(v) the dried granulation is de-lumped using a suitable mill (such as Comill or Fitzmill);
(vi) the de-lumped granules are blended with an extragranular portion of crospovidone (equal to 3% of the weight of the pioglitazone layer) in a suitable blender to give a granulation lend; and
(vii) the granulation lend is lubricated with sodium stearyl fumarate in a suitable blender.
   (3) Preparation of the Bilayer Tablet via bilayer compression and coating:
(i) the Sitagliptin layer and Pioglitazone layer are compressed into a bilayer tablet in a suitable bilayer tableting machine; and
(ii) the bilayer tablet is coated with Opadry I (HPC/HPMC) or Opadry II HP (PVA-PEG) or Opadry II (PVA-HPMC) coating system to the desired weight gain.

The first layer (the DPP-4 inhibitor layer) can be the layer at the bottom of the bilayer tablet or at the top of the bilayer tablet (filled into the die either first or second). The second layer (the pioglitazone layer) can be the layer at the bottom of the bilayer tablet or at the top of the bilayer tablet (filled into the die either first or second).

The present invention provides a fixed dose combination of a dipeptidyl peptidase-4 (DPP-4) inhibitor, or a pharmaceutically acceptable salt thereof, and pioglitazone, or a pharmaceutically acceptable salt thereof; in which both drugs are stable in a single tablet. More particularly, the present invention provides a fixed dose combination comprised of a layer of a dipeptidyl peptidase-4 (DPP-4) inhibitor, or a pharmaceutically acceptable salt thereof, and a layer of pioglitazone HCl in a single bilayer tablet, in which the conversion of pioglitazone HCl to the pioglitazone free base via disproportionation is minimized.

The present invention also provides methods for treating Type 2 diabetes by orally administering to a host in need of such treatment a therapeutically effective amount of one of the fixed-dose combination pharmaceutical compositions of the present invention. In one embodiment the host in need of such treatment is a human. In another embodiment the pharmaceutical composition is in the dosage form of a tablet. The pharmaceutical compositions comprising the fixed-dose combination may be administered once-daily (QD), twice-daily (BID), or thrice-daily (TID).

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### Example 1

### Fixed-Dose Combination of 100 Milligrams Sitagliptin and 15 Milligrams Pioglitazone Per Bilayer Tablet

| | |
|---|---|
| Formulation Ingredients | 100/15 mg |

Pioglitazone Layer (mg/tablet)

| Pioglitazone hydrochloride** | 16.53 | mg |
|---|---|---|
| Lactose monohydrate | 71.47 | mg |
| Hydroxypropyl cellulose | 3 | mg |
| Crospovidone | 8 | mg |
| Sodium stearyl fumarate | 1 | mg |
| Purified water for | 47 mg containing 3 mg | |
| granulation step*** | HPC for fluid bed | |
| Pioglitazone layer weight | 100 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Dibasic calcium phosphate, | 123.75 | mg |
| Anydrous | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 500 | mg |
| Opadry 20A18334 | 15 | mg |
| (HPC/HPMC) coating | | |
| Purified water for | 135 | mg |
| coating step*** | | |
| Total coated tablet weight | 515 | mg |

*Equivalent to 100 mg of sitagliptin free base anhydrate.
**Equivalent to 15 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and a portion of crospovidone (62.5% of total amount) were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone (37.5%) in the V-shell blender for 10 minutes. Sodium stearyl fumarate was sieved through #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 515 mg coated tablet.

### Example 2

### Fixed-Dose Combination of 100 Milligrams Sitagliptin and 30 Milligrams Pioglitazone Per Bilayer Tablet

| | |
|---|---|
| Formulation Ingredients | 100/30 mg |

Pioglitazone Layer (mg/tablet)

| Pioglitazone hydrochloride** | 33.06 | mg |
|---|---|---|
| Lactose monohydrate | 142.94 | mg |
| Hydroxypropyl cellulose | 6 | mg |
| Crospovidone | 16 | mg |
| Sodium stearyl fumarate | 2 | mg |
| Purified water for | 94 mg containing 6 mg | |
| granulation step*** | HPC for fluid bed | |
| Pioglitazone layer weight | 200 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Dibasic calcium phosphate, | 123.75 | mg |
| Anydrous | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 600 | mg |
| Opadry 20A18334 coating | 18 | mg |
| Purified water for | 162 | mg |
| coating step*** | | |
| Total coated tablet weight | 618 | mg |

*Equivalent to 100 mg of sitagliptin free base anhydrate.
**Equivalent to 30 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and a portion of crospovidone (62.5% of total amount) were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone (37.5%) in the V-shell blender for 10 minutes. Sodium stearyl fumarate was sieved through #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 618 mg coated tablet.

### Example 3

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 214.41 | mg |
| Hydroxypropyl cellulose | 9 | mg |
| Crospovidone | 24 | mg |
| Sodium stearyl fumarate | 3 | mg |
| Purified water for | 141 mg containing 9 mg | |
| granulation step*** | HPC for fluid bed | |
| Pioglitazone layer weight | 300 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Dibasic calcium phosphate, | 123.75 | mg |
| Anydrous | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 700 | mg |
| Opadry 20A18334 | 21 | mg |
| (HPC/HPMC) coating OR | | |
| Opadry 85F96652 (PVA/PEG) | | |
| coating OR Opadry 89F | | |
| (PVA/HPMC) coating | | |
| Purified water for | 189 | mg |
| coating step*** | | |
| Total coated tablet weight | 721 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and a portion of crospovidone (62.5% of total amount) were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone (37.5%) in the V-shell blender for 10 minutes. Sodium stearyl fumarate was sieved though #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 721 mg coated tablet.

### Example 4

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 223.4 | mg |
| Hydroxypropyl cellulose | 9 | mg |
| Crospovidone | 30.8 | mg |
| Sodium stearyl fumarate | 3.16 | mg |
| Purified water for | 141 mg containing 9 mg | |
| granulation step*** | HPC for fluid bed | |
| Pioglitazone layer weight | 100 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Anhydrous Dibasic calcium | 123.75 | mg |
| phosphate | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 500 | mg |
| Opadry 20A18334 | 21.5 | mg |
| (HPC/HPMC) coating | | |
| Purified water for | 193.5 | mg |
| coating step*** | | |
| Total coated tablet weight | 737.4 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and half of crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone in the V-shell blender for 10 minutes. Sodium stearyl fumarate was sieved through #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 737.4 mg coated tablet.

### Example 5

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 220.41 | mg |
| Hydroxypropyl cellulose | 9 | mg |
| Crospovidone | 9 | mg |
| Magnesium stearate | 3 | mg |
| Purified water for | 141 mg containing 9 mg | |
| granulation step*** | HPC for fluid bed | |
| Pioglitazone layer weight | 291 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Anhydrous Dibasic calcium | 123.75 | mg |
| phosphate | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 691 | mg |
| Opadry I (HPC/HPMC) coating | 21 | mg |
| Purified water for | 189 | mg |
| coating step*** | | |
| Total coated tablet weight | 712 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. Magnesium stearate was sieved through #60 sieve. The sieved magnesium stearate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 712 mg coated tablet.

### Example 6

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 223.41 | mg |
| Hydroxypropyl cellulose | 9 | mg |
| Crospovidone | 15 | mg |
| Sodium stearyl fumarate | 3 | mg |
| Purified water for | 141 mg containing 9 mg | |
| granulation step*** | HPC for fluid bed | |
| Pioglitazone layer weight | 300 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Anhydrous Dibasic calcium | 123.75 | mg |
| phosphate | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 700 | mg |
| Opadry I (HPC/HPMC) coating | 21 | mg |
| Purified water for | 189 | mg |
| coating step*** | | |
| Total coated tablet weight | 721 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. Sodium stearyl fumarate was sieved through #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 721 mg coated tablet.

### Example 7

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 217.41 | mg |
| Hydroxypropyl cellulose | 15 | mg |
| Crospovidone | 15 | mg |
| Sodium stearyl fumarate | 3 | mg |
| Purified water for | 235 mg containing 15 mg | |
| granulation step*** | HPC for fluid bed | |
| Pioglitazone layer weight | 300 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Anhydrous Dibasic calcium | 123.75 | mg |
| phosphate | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 700 | mg |
| Opadry I (HPC/HPMC) coating | 21 | mg |
| Purified water for | 189 | mg |
| coating step*** | | |
| Total coated tablet weight | 721 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. Sodium stearyl fumarate was sieved through #60 sieve. The sieved sodium stearyl fumarate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 721 mg coated tablet.

### Example 8

### Fixed-Dose Combination of 100 Milligrams Sitagliptin and 30 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 221.91 | mg |
| Hydroxypropyl cellulose | 9 | mg |
| Crospovidone | 18 | mg |
| Magnesium stearate | 1.5 | mg |
| Purified water for | 141 mg containing 9 mg | |
| granulation step*** | HPC for fluid bed | |
| Pioglitazone layer weight | 300 | mg |

Sitagliptin Layer (mg/tablet)

| | | |
|---|---|---|
| Sitagliptin Phosphate | 128.5 | mg |
| Monohydrate* | | |
| Microcrystalline cellulose | 123.75 | mg |
| Anhydrous Dibasic calcium | 123.75 | mg |
| phosphate | | |
| Croscarmellose sodium | 8 | mg |
| Magnesium stearate | 4 | mg |
| Sodium stearyl fumarate | 12 | mg |
| Sitagliptin layer weight | 400 | mg |
| Total core tablet weight | 700 | mg |
| Opadry I (HPC/HPMC) coating | 21 | mg |
| Purified water for | 189 | mg |
| coating step*** | | |
| Total coated tablet weight | 721 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, anhydrous dibasic calcium phosphate and croscarmellose sodium were blended in a bin blender for 10 minutes. Sodium stearyl fumarate and magnesium stearate were sieved through #60 sieve. The sieved sodium stearyl fumarate and magnesium stearate were blended with the sitagliptin blend for additional 5 minutes to give the lubricated sitagliptin powder blend.

Preparation of the layer containing Pioglitazone (the Pioglitazone Granulation Layer):

Pioglitazone hydrochloride, lactose and half of crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone in the V-shell blender for 10 minutes. Magnesium stearate was sieved through #60 sieve. The sieved magnesium stearate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 721 mg coated tablet.

### Example 9

### Fixed-Dose Combination of 100 Milligrams Sitagliptin and 45 Milligrams Pioglitazone Per Bilayer Tablet

| | |
|---|---|
| Formulation Ingredients | 100/45 mg |

Pioglitazone Layer (mg/tablet)

| Pioglitazone hydrochloride** | 49.59 | mg |
|---|---|---|
| Lactose monohydrate | 221.91 | mg |
| Hydroxypropyl cellulose | 9 | mg |
| Crospovidone | 18 | mg |
| Magnesium stearate | 1.5 | mg |
| Purified water for | 141 mg containing 9 mg | |
| granulation step*** | HPC for fluid bed | |
| Pioglitazone layer weight | 300 | mg |

Sitagliptin Layer (mg/tablet)

| | | |
|---|---|---|
| Sitagliptin Phosphate | 128.5 | mg |
| Monohdyrate* | | |
| Microcrystalline cellulose | 121.75 | mg |
| Mannitol | 121.75 | mg |
| Crospovidone | 12 | mg |
| Magnesium stearate | 4 | mg |
| Sodium stearyl fumarate | 12 | mg |
| Sitagliptin layer weight | 400 | mg |
| Total core tablet weight | 700 | mg |
| Opadry I (HPC/HPMC) coating | 21 | mg |
| Purified water for | 189 | mg |
| coating step*** | | |
| Total coated tablet weight | 721 | mg |

*Equivalent to 100 mg of sitagliptin free base anhydrate.
**Equivalent to 45 mg of pioglitazone free base.
***Removed during processing.

Method of Manufacture:

Preparation of the layer containing Sitagliptin (the Sitagliptin Powder Blend Layer): Sitagliptin phosphate monohydrate, microcrystalline cellulose, mannitol and crospovidone were blended in a bin blender for 10 minutes. Magnesium stearate and sodium stearyl fumarate were sieved through #60 sieve. The powder mixture was blended with magnesium stearate for 5 minutes and roller compacted in Alexanderwek roller compactor. The resulting ribbons were milled through rotary fine granulator attachment on the roller compactor to give roller compacted granules. The granules were blended with sodium stearyl fumarate in the bin blender to give the lubricated sitagliptin granulation.

Preparation of the layer containing Pioglitazone (the Pioglitazone Granulation Layer):

Pioglitazone hydrochloride, lactose and half of crospovidone were blended in a V-shell blender for 10 minutes. The mixture was de-lumped through a co-mill. The de-lumped mixture was loaded into a fluid bed granulator. The mixture was granulated using 6% w/w solution of hydroxypropyl cellulose in the fluid bed granulator. The wet mass was dried in the fluid bed granulator. The dried granulation was de-lumped using a co-mill to achieve uniform granules. The milled granules were blended with the remaining crospovidone in the V-shell blender for 10 minutes. Magnesium stearate was sieved through #60 sieve. The sieved magnesium stearate was blended with the pioglitazone granulation for additional 5 minutes to give the lubricated pioglitazone granulation blend.

Compression and Formation of the Bilayer Tablet:

The bilayer tablets were compressed on the Piccola bilayer tablet press using sitagliptin powder blend as the first layer and pioglitazone granulation as the second layer. The tablets were film coated with a suitable Opadry® suspension (such as Opadry® 20A18334) to an approximate 3% weight gain to provide a 721 mg coated tablet.

What is claimed is:

1. A pharmaceutical composition in the form of a bilayer tablet comprising:
(a) a first layer comprising about 20 to 45% by weight of a dipeptidyl peptidase-4 inhibitor, or a pharmaceutically acceptable salt thereof; and
(b) a second layer comprising about 7 to 24% by weight of pioglitazone hydrochloride.

2. The pharmaceutical composition of claim 1 wherein the first layer additionally comprises one or more excipients selected from the group consisting of: (i) a diluent; (ii) a disintegrant; and (iii) a lubricant.

3. The pharmaceutical composition of claim 2 wherein the first layer additionally comprises a binding agent.

4. The pharmaceutical composition of claim 1 wherein the first layer additionally comprises excipients selected from the group consisting of: (i) about 40-80% by weight of a diluent; (ii) about 0.5-6% by weight of a disintegrant; and (iii) about 0.75-10% by weight of a lubricant.

5. The pharmaceutical composition of claim 1 wherein the second layer additionally comprises one or more excipients selected from the group consisting of (i) a diluent, (ii) a disintegrant; (iii) a binding agent; and (iv) a lubricant.

6. The pharmaceutical composition of claim 1 wherein the second layer additionally comprises excipients selected from the group consisting of: (i) about 60-80% by weight of a diluent; (ii) about 2-12% by weight of a disintegrant; and (iii) about 1-7% by weight of a binding agent; and (iv) about 0.25-4% by weight of a lubricant.

7. The pharmaceutical composition of claim 1 comprising:
(a) a first layer comprising:
(i) about 20 to 45% by weight of a dipeptidyl peptidase-4 inhibitor, or a pharmaceutically acceptable salt thereof;
(ii) about 40-80% by weight of a diluent;
(iii) about 0.5-6% by weight of a disintegrant; and
(iv) about 0.75-10% by weight of a lubricant; and
(b) a second layer comprising:
(i) about 7 to 24% by weight of pioglitazone hydrochloride;
(ii) about 60-80% by weight of a diluent;
(iii) about 2-12% by weight of a disintegrant;
(iv) about 1-7% by weight of a binding agent, and
(v) about 0.25-4% by weight of a lubricant.

8. The pharmaceutical composition of claim 7 wherein the diluent in the first layer is selected from the group consisting of microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof.

9. The pharmaceutical composition of claim 7 wherein the diluent in the first layer is a mixture of microcrystalline cellulose and mannitol; the disintegrant is crospovidone; and the lubricant is a mixture of magnesium stearate and sodium stearyl fumarate.

10. The pharmaceutical composition of claim 7 wherein the diluent in the first layer is a mixture of microcrystalline cellulose and anhydrous dibasic calcium phosphate; the disintegrant is croscarmellose sodium; and the lubricant is a mixture of magnesium stearate and sodium stearyl fumarate.

11. The pharmaceutical composition of claim 7 wherein the diluent in the second layer is selected from the group consisting of: lactose monohydrate, microcrystalline cellulose and mannitol, or a mixture thereof; the disintegrant is selected from the group consisting of: crospovidone and croscarmellose sodium, or a mixture thereof; the binding agent is hydroxypropyl cellulose; and the lubricant is selected from the group consisting of magnesium stearate, and sodium stearyl fumarate, or a mixture thereof.

12. The pharmaceutical composition of claim 7 wherein the diluent in the second layer is lactose monohydrate; the disintegrant is crospovidone; the binding agent is hydroxypropyl cellulose; and the lubricant is magnesium stearate.

13. The pharmaceutical composition of claim 7 wherein the diluent in the second layer is lactose monohydrate; the disintegrant is crospovidone; the binding agent is hydroxypropyl cellulose; and the lubricant is sodium stearyl fumarate.

14. The pharmaceutical composition of claim 1 wherein the dipeptidyl peptidase-4 inhibitor is selected from the group consisting of alogliptin, carmegliptin, denagliptin, dutogliptin, linagliptin, melogliptin, saxagliptin, sitagliptin, and vildagliptin, or a pharmaceutically acceptable salt of each thereof.

15. The pharmaceutical composition of claim 1 wherein the dipeptidyl peptidase-4 inhibitor is sitagliptin, or the dihydrogenphosphate salt thereof.

16. The pharmaceutical composition of claim 1 comprising:
(a) a first layer comprising:
(i) about 25 to 35% by weight of a dipeptidyl peptidase-4 inhibitor, or a pharmaceutically acceptable salt thereof;
(ii) about 50-70% by weight of a diluent;
(iii) about 1-4% by weight of a disintegrant; and
(iv) about 1.5-7% by weight of a lubricant; and
(b) a second layer comprising:
(i) about 12 to 20% by weight of pioglitazone hydrochloride;
(ii) about 65-75% by weight of a diluent;
(iii) about 3-11% by weight of a disintegrant;
(iv) about 2-5% by weight of a binding agent; and
(v) about 0.5-2.5% by weight of a lubricant.

17. The pharmaceutical composition of claim 16 wherein the dipeptidyl peptidase-4 inhibitor in the first layer is sitagliptin, or a pharmaceutically acceptable salt thereof; the diluent is a mixture of microcrystalline cellulose and mannitol, or a mixture of microcrystalline cellulose and anhydrous dibasic calcium phosphate; the disintegrant is croscarmellose sodium or crospovidone; and the lubricant is selected from the group consisting of magnesium stearate, and sodium stearyl fumarate, or a mixture thereof.

18. The pharmaceutical composition of claim 16 wherein the diluent in the second layer is lactose monohydrate; the disintegrant is crospovidone; the binding agent is hydroxypropylcellulose; and the lubricant is magnesium stearate.

19. The pharmaceutical composition of claim 16 wherein the diluent in the second layer is lactose monohydrate; the disintegrant is crospovidone; the binding agent is hydroxypropylcellulose; and the lubricant is sodium stearyl fumarate.

20. The pharmaceutical composition of claim 16 wherein the dipeptidyl peptidase-4 inhibitor is present in a unit dosage strength of 25, 50, 75, 100, 150, or 200 milligrams, and the pioglitazone is present in a unit dosage strength of 15, 30, or 45 milligrams.

21. The pharmaceutical composition of claim 16 wherein the dipeptidyl peptidase-4 inhibitor is sitagliptin, or a pharmaceutically acceptable salt thereof.

22. The pharmaceutical composition of claim 21 wherein the sitagliptin is present in a unit dosage strength of 50 or 100 milligrams, and the pioglitazone is present in a unit dosage strength of 15, 30 or 45 milligrams.

23. The pharmaceutical composition of claim 1 wherein said composition is in the dosage form of a tablet.

24. A method of treating Type 2 diabetes in a human in need thereof comprising orally administering to said human a pharmaceutical composition of claim 1.

25. The pharmaceutical composition of claim 1 further comprising one or more agents selected from the group consisting of flavoring agents, colorants, and sweeteners.

26. The pharmaceutical composition of claim 1 wherein the bilayer tablet is coated with a film-coating agent.

27. The pharmaceutical composition of claim 1 wherein said DPP-4 inhibitor is vildagliptin, or a pharmaceutically acceptable salt of each thereof.

28. The pharmaceutical composition of claim 1 wherein said DPP-4 inhibitor is saxagliptin, or a pharmaceutically acceptable salt of each thereof.

29. The pharmaceutical composition of claim 1 wherein said DPP-4 inhibitor is alogliptin, or a pharmaceutically acceptable salt of each thereof.
